(19) United States
(12) Patent Application Publication
(10) Pub. No.: US 2020/0199575 A1
(43) Pub. Date: Jun. 25, 2020

(54) ROLE OF EXOSOMES, EXTRACELLULAR VESICLES, IN THE REGULATION OF METABOLIC HOMEOSTASIS

(71) Applicant: CHILDREN'S HOSPITAL MEDICAL CENTER, Cincinnati, OH (US)

(72) Inventor: Takahisa NAKAMURA, Cincinnati, OH (US)

(21) Appl. No.: 16/807,260

(22) Filed: Mar. 3, 2020

Related U.S. Application Data
(63) Continuation of application No. PCT/US2018/050180, filed on Sep. 10, 2018.
(60) Provisional application No. 62/558,024, filed on Sep. 13, 2017.

(51) Int. Cl. C12N 15/10; A61P 3/08

(52) U.S. Cl. CPC C12N 15/1017; C12N 2320/30; C12N 2310/141; A61P 3/08

(57) ABSTRACT

A method is described for regulating metabolic homeostasis in a subject in need thereof, the method comprising isolating circulating exosomes from a healthy donor, and administering the exosomes to the subject, under conditions sufficient to regulate metabolic homeostasis in the subject, wherein the exosomes act by a direct interaction with insulin target tissues and/or by modulation of immune function. Furthermore, the components of the exosomes, such as RNAs and/or products catalyzed by sphingomyelin phosphodiesterase 3 (SMPD3) are used to treat metabolic disorders, such as insulin resistance and type 2 diabetes. In addition, a method for early detection of metabolic risks associated with obesity is provided.

### CROSS-REFERENCE TO RELATED APPLICATIONS

This application is a continuation of International Application No. PCT/US2018/050180 filed on Sep. 10, 2018, which claims priority to U.S. Provisional Patent Application No. 62/558,024 filed on Sep. 13, 2017. The entire contents of each of these applications is incorporated by reference herein.

### FIELD OF THE INVENTION

Provided is a method for regulating metabolic homeostasis in a subject in need thereof, the method comprising isolating circulating exosomes from a healthy donor, and administering the exosomes to the subject, under conditions sufficient to regulate metabolic homeostasis in the subject. Further provided is a method for early detection of metabolic risks associated with obesity, the method comprising obtaining circulating exosomes from a subject, and measuring a concentration of exosomes and/or components of the exosomes.

### BACKGROUND

The worldwide prevalence of obesity has reached pandemic proportions, bringing with it a host of metabolic problems such as insulin resistance and type 2 diabetes (T2D). According to the World Health Organization, T2D will affect >300 million people by the year 2025. Despite enormous research efforts put forth in the study of obesity and diabetes over the past 30 years, the molecular mechanisms by which obesity leads to insulin resistance and T2D remain elusive.

Bariatric surgery, the most successful intervention for severe obesity, results in rapid and marked improvement of insulin resistance and type 2 diabetes as early as the first week post-surgery, prior to significant weight loss. However, the molecular mechanisms of this rapid and weight-loss-independent antidiabetic effect are not clearly understood.

In addition, accumulated evidence indicates that the development of insulin resistance and T2D in obesity has strong inflammatory underpinnings. Significant activation and infiltration of immune cells into metabolic tissues, such as the liver, lead to chronic activation of inflammatory pathways in both tissues and immune cells. These abnormal events, in turn, trigger stress kinase activation that impinges on the normal signaling of metabolic hormones such as insulin, subsequently impairing glucose metabolism. However, how immune cells in obese conditions are activated, remains poorly understood.

Extracellular vesicles (EVs) including microvesicles (30-1000 nm) and exosomes (30-100 nm), that are released from many cell types into the extracellular space, are distributed in body fluids. These EVs are taken up by neighboring or distant cells and subsequently modulate functions of the recipient cells. EVs are composed of lipid bilayer membrane containing various nucleic acids, proteins, and lipids in the lumen. The components of EVs reflect those of their cellular origin. Therefore, when the secreting cells are under abnormal environments, the compositional changes are reflected in the EVs, thereby sending the wrong messages to the recipient cells. Indeed, it has been demonstrated that EVs secreted from cancer cells exert higher pro-inflammatory effects on recipient immune cells and induce inflammation. These observations suggest the existence of pathogenic EVs secreted by the pathological cells. As obesity causes drastic changes in expressions of RNAs, proteins, and lipids in metabolic cells such as hepatocytes, EVs secreted from such cells may take on pathogenic characteristics and inappropriately activate immune cells locally and systemically.

In addition, insulin resistance is a significant co-morbidity of severe obesity, and serves as a major risk factor for metabolic complications. Despite its significant clinical relevance and high prevalence, the pathophysiology underlying insulin resistance is not fully understood.

We have critically evaluated the role of obesity-driven, hepatocyte-derived extracellular vesicles, including post-bariatric surgery, in the regulation of inflammation and glucose metabolism, utilizing our unique mouse model that permits monitoring of tissue-specific extracellular vesicles in vivo. Based on our results, described in detail below, we have developed novel extracellular vesicles-based therapeutic options for insulin resistance and T2D, towards reducing the burden of pandemic obese issues.

### SUMMARY

As described in detail below, under certain conditions such as obesity, parenchymal cells including hepatocytes secret pro-inflammatory, pathogenic extracellular vesicles including exosomes and trigger abnormal inflammation, leading to obesity-associated sequelae such as type 2 diabetes, non-alcoholic steatohepatitis (NASH) and cancer. Therefore, in one aspect, these pathogenic extracellular vesicles are therapeutic targets.

In one aspect, a method for treating metabolic disorders in a subject in need thereof is provided, where the method comprises isolating circulating extracellular vesicles (EV) from a healthy donor or from media of cultured cells, and administering the EVs to the subject, under conditions sufficient to treat metabolic disorders in the subject. In one embodiment, the metabolic disorders is an obesity-associated metabolic disease selected from insulin resistance, type-2 diabetes, fatty liver diseases, cardiovascular disease, atherosclerosis, and/or Alzheimer's disease. In one embodiment, the EVs comprise miR-191, miR-150, LINC00237, and/or SMPD3. In one embodiment, the EVs act by a direct interaction with insulin target tissues and/or by modulation of immune function. In various embodiments, the insulin target tissues are selected from the group consisting of liver, adipose tissue, muscle, and combinations thereof In one embodiment, the EVs are isolated by an affinity-based isolation procedure using a phosphatidylserine (PS)-binding protein or by size-exclusion chromatography. In one embodiment, insulin resistance is measured by measuring blood glucose, hemoglobin A1c, and/or insulin, where the measurements are conducted before and after the administration of the EVs. In one embodiment, the method further comprises assaying the effect on immune function by measuring circulating levels of cytokines selected from the group consisting of IL-6, TNF-α, and IL-10 before and after the administration of the exosomes. In one embodiment, the exosomes are administered at a dose and/or concentration similar to levels in healthy subjects. In one embodiment, the healthy donor exhibits at least one of a body mass index (BMI) of less than about 25, normal blood glucose level, and/or no symptoms of fatty liver disease. In one embodiment, when the EVs are derived from cultured cells, the cultured cells are hepatocytes. In one embodiment, the EV is an exosome.

In another aspect, a method for early detection of metabolic risks associated with obesity is provided. In one embodiment, the method comprises obtaining circulating extracellular vesicles (EVs) from a subject, measuring a concentration of EVs and/or a level of miR-191, miR-150, LINC00237, and/or sphingomyelin phosphodiesterase 3 (SMPD3) in the EVs, comparing the concentration of EVs and/or the level of miR-191, miR-150, LINC00237, and/or SMPD3 in the EVs with the EV concentration and/or level of miR-191, miR-150, LINC00237, and/or SMPD3 in EVs from a healthy control, whereby i) if the concentration of the EVs is greater than the healthy control; ii) if the level of miR-191 is greater than the healthy control; iii) if the level of miR-150 is less than the healthy control; iv) if the level of LINC00237 is less than the healthy control; v) if the level of SMPD3 is greater than the healthy control, the subject exhibits metabolic risks associated with obesity. In one embodiment, the method further comprises treating the subject found to exhibit metabolic risks associated with obesity. In one embodiment, the treatment comprises neutralizing or depleting an amount of the EVs, or a subset thereof, such that development of metabolic diseases is ameliorated or prevented. In various embodiments, the metabolic risks comprise an obesity-associated metabolic disease selected from insulin resistance, type-2 diabetes, fatty liver diseases, cardiovascular disease, atherosclerosis, and/or Alzheimer's disease.

In yet another aspect, a method for treating insulin resistance and/or type-2 diabetes is provided. In one embodiment, the method comprises administering an inhibitor of sphingomyelin phosphodiesterase 3 (SMPD3) to a subject in need thereof, under conditions sufficient to treat insulin resistance and/or type-2 diabetes. In one embodiment, the inhibitor of sphingomyelin phosphodiesterase 3 (SMPD3) is GW4869, metformin, or siRNA directed to SMPD3 mRNA. In one embodiment, metformin decreases the expression of SMPD3. In one embodiment, SMPD3 stimulates ceramide generation and RNA loading into extracellular vesicles (EVs). In one embodiment, inhibition of SMPD3 reduces pro-inflammatory exosome secretion, decreases local and systemic inflammation, and improves glucose metabolism.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an illustration of current findings. (MH: Metabolically healthy, MU: Metabolically unhealthy, IS: Insulin sensitive, IR: Insulin resistance)

FIG. 2 shows illustrations of how exosomes serve as a novel biomarker or a therapeutic target. (A) Schematics of two different types of exosome. Blue indicates metabolically healthy components and red indicates metabolically unhealthy components. Green indicates exosome components that are not associated with metabolic conditions. Black arrows pointing at potential therapeutic targets, including proteins and nucleic acids. (B) Exosomes as a biomarker capable to detect early molecular changes prior to disease manifestation and/or variable metabolic risks between different obese individuals.

FIG. 3 shows mean exosome concentration (blue) and mean BMI (red) at pre-op, 1, 3 and 6 months post-op. Blood samples were drawn after 8 hour fasting at each time point. Data are shown as the mean±SEM. (*p<0.05, t-test performed for exosome concentration between each time point).

FIG. 4 shows silver staining of exosomal protein (n=4). A red (top) arrow pointing at a group of proteins with an upregulation, and a yellow (bottom) arrow pointing at a group of proteins with a downregulation 1 month post-op. (Pre: before surgery, Post; 1 month post-op)

FIG. 5 shows a heatmap for exosomal RNA-seq data. Red-boxed RNAs are previously associated with immune/metabolic function. (n=10, Pre: before surgery, Post; 1 months after surgery).

FIG. 6 shows (A) an illustration of the affinity binding exosome purification method (WAKO). (B) an example of the NanoSight analysis of exosomes isolated from an obese individual before bariatric surgery. (C) a TEM image of exosomes isolated from an obese adolescent, with the typical exosome size of 30-150 nm and signature central depression.

FIG. 7 shows EVs secreted from hepatocytes in obesity take on the pathogenic characteristics and activate immune cells locally and systemically, leading to insulin resistance and T2D.

FIG. 8 shows functional assay of exosomes utilizing human monocytes. The data shows that exosomes from a lean and metabolically healthy individual suppress cytokine secretion in a physiologic setting (PBS), but exaggerate immune response when there is a pathogen (LPS). A cell to exosome ratio of 1:5 was used for this experiment. (Exo: exosomes).

FIG. 9 shows the establishment of a novel mouse model monitoring tissue-specific EVs in vivo. (A) An in vivo strategy to label EVs with GFP in a tissue-specific manner. GFP fused with EV target sequence (ETS-GFP) can be expressed under the control of Cre-recombinase. (B) By crossing ETS-GFP mice with Albumin-cre mice (ETS-GF^{Alb-cre}), GFP signal was detected in the liver section. (C) One example of GFP-labeled EV transfer from hepatocytes to other cells. After removal of hepatocytes from the liver of ETS-GFP^{Alb-cre} mice, non-parenchymal cells were subjected to the flow cytometry analysis (FCM). GFP signal in non-parenchymal cells was detected. (D) Results of ImageStreamX analysis. Immune cells, including monocytes and T-cells, engulf GFP-labeled EV.

FIG. 10 shows SMPD3 levels in obesity. Expression levels of SMPD3 in the liver of lean (regular diet (RD) on 33 weeks, n=8) and obese (high fat diet (HFD) on 33 weeks, n=6) mice (C57BL/6J). **p<0.01.

FIG. 11 shows pro-inflammatory effects of hepatocyte-derived exosomes on macrophages. (A) A scheme of the exosome transfer from cultured primary mouse hepatocytes to primary mouse bone marrow-derived macrophages (BMDMs). (B) One example showing pro-inflammatory effects of exosomes isolated from hepatocytes. Exosomes isolated from hepatocytes treated with palmitate (PA: 100 μM) for 20 hours increase expression of IL-6 mRNA in BMDMs. Conversely, exosomes collected from hepatocytes cotreated with PA with metformin (Met: 250 μM) and/or SMPD3 inhibitor (GW: GW4869: 10 μM) for 20 hours alleviate IL-6 expression. The same number of exosomes was used. As there is no additive effect of metformin and SMPD3 inhibitor, metformin may exert its anti-inflammatory effect by suppressing SMPD3 function. (C) Expression levels of SMPD3 are suppressed in primary hepatocytes by metformin treatment for 24 hours. *p<0.05, **p<0.01.

### DETAILED DESCRIPTION

In one aspect, we have shown that exosomes, a novel mode of intercellular communication, play a critical role in the rapid and profound improvement of insulin sensitivity after bariatric surgery. Exosomes are nano-sized extracellular vesicles secreted by different cell types. Exosomes are enclosed by bilayer membranes, and contains various proteins and nucleic acids, including microRNAs (miRNAs) and long non-coding RNAs (lncRNAs). These vesicles travel to neighboring and distant organs, and subsequently modulate the cellular functions within the recipient cells. Essentially, exosomes serve as a novel mode of intercellular communication. Additional evidence suggests that exosomes play a key role in pathophysiological processes, including obesity and its metabolic complications, and can serve as novel biomarkers and even therapeutic targets.

We have observed a significant reduction in total serum exosome concentration and altered exosomal RNA expression pattern in obese adolescents one month after bariatric surgery. Importantly, these changes happen in the first month after bariatric surgery when the patients experience marked improvement of insulin sensitivity prior to the significant weight loss. Additionally, recent studies in animals demonstrate that exosomes modulate insulin sensitivity. For example, exosomes from obese mice induce insulin resistance in lean mice and exosomes from lean mice improve insulin resistance in obese mice. The suggested mechanism is regulation of gene expression at their target tissues via transferring exosomal miRNAs. The current data indicate more than one pathway by which exosomes can regulate insulin sensitivity; 1) a direct interaction with insulin target tissues, such as liver, adipose tissue or muscle, and 2) modulation of immune function. The observation of an immune-modulating property of exosomes is highly relevant as accumulating evidences for the past two decades support an inextricable link between obesity, aberrant inflammation and metabolic co-morbidities, including insulin resistance. Based on these novel observations, bariatric surgery promotes improvement of insulin sensitivity, at least in part, by modulating serum exosome profile.

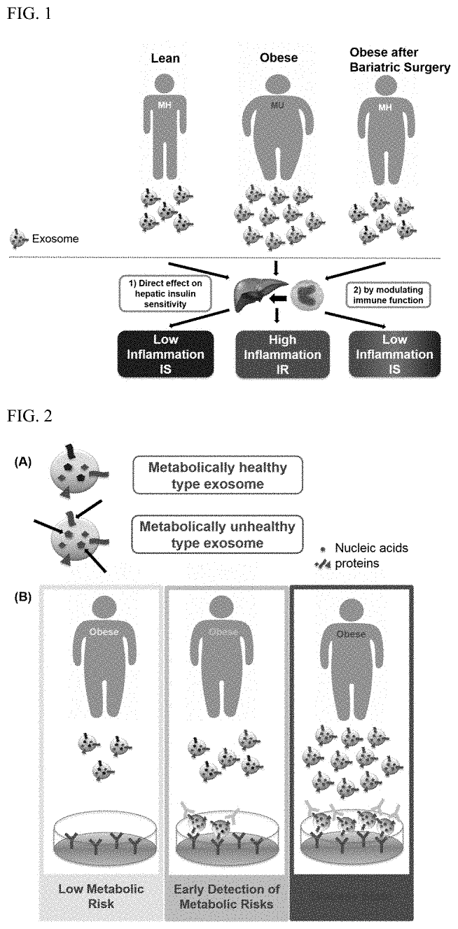

We designed a prospective longitudinal study to perform comprehensive analyses of serum exosome profile isolated from obese adolescents before, 1 and 3 months after bariatric surgery (FIG. 1). We chose to focus on the immediate period after the surgery, to highlight weight-loss-independent mechanisms. Our methodology includes both quantitative (serum concentration) and compositional (morphology and exosomal protein and RNA) analyses of exosomes.

By defining the role of exosomes in the improvement of insulin sensitivity after bariatric surgery, we will further our understanding of the pathophysiology underlying insulin resistance. This novel mechanism has the potential for broad application in human disease mediated by insulin resistance, such as a sensitive biomarker for early detection of metabolic risks associated with obesity (FIG. 2A). Additionally, the unique exosomal components, such as proteins or nucleic acids, play a key role in the regulation of insulin sensitivity, and therefore, these molecules can serve as a target for novel treatments that are less-invasive yet as effective as bariatric surgery (FIG. 2A).

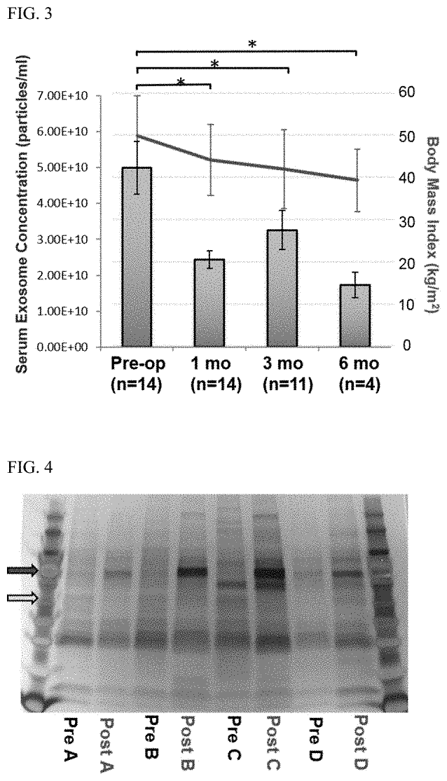

In our study, we observed a significant reduction in serum exosome concentration 1 month after surgery, and this trend appears to persist up to 6 months post-op (FIG. 3). Concurrently, the analyses of exosome contents, including proteins and RNAs, was performed. First, we evaluated protein contents of exosomes by performing silver staining utilized exosome samples obtained pre-op and 1 month post-op. This simple experiment uncovered a distinctive pattern of exosomal proteins, of which some are upregulated and some are downregulated 1 month after surgery (FIG. 4, n=4).

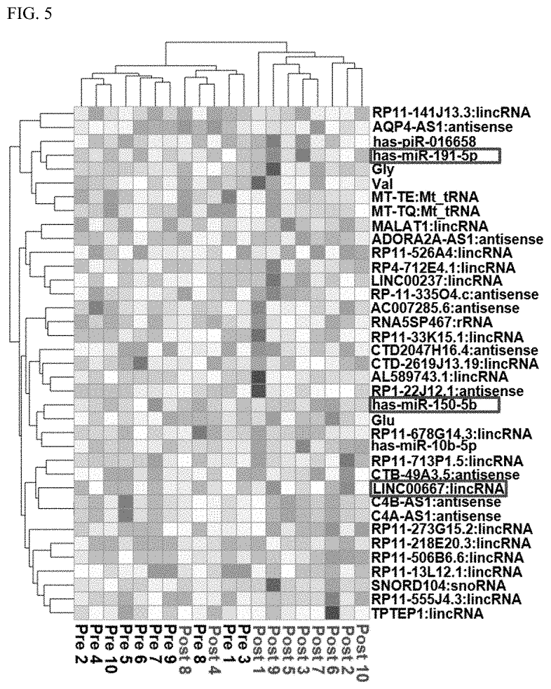

To further investigate the exosome contents, we extracted RNAs from exosomes isolated from 500 μl of serum samples from study subjects before and 1 month after surgery. Subsequently, we performed RNA deep sequencing (n=10). While exosomal RNA sequencing (RNA-seq) has been challenging due to the small RNA amount within exosomes, a novel whole RNA deep sequencing technique for exosomal RNAs has been developed (U.S. Patent Application No. 62/434,531, incorporated herein in its entirety). With this technique, we successfully completed whole exosomal RNA-seq. Bioinformatic analysis of the RNA-seq data showed that the profile of a number of different RNA species, including miRNAs and lncRNAs, associated with obesity, glucose metabolism and immune function, change 1 month after surgery (FIG. 5). Among these exosomal RNAs that are differentially expressed 1 month after surgery, we identified several candidates that may regulate insulin sensitivity and/or immune function; e.g., miR-191 that is downregulated by 1.6-fold was previously shown to directly interact with insulin receptor substrate 1 (Irs1), miR-150 that is upregulated by 1.95 folds has been shown to target AKT (Protein kinase B) and modulates immune function, and LINC00237 that is upregulated by 5 folds has been associated with obesity (p value<0.05).

Importantly, these changes happen during the first month after surgery, when a major improvement of insulin sensitivity is achieved concurrent with a modest initial weight loss (FIG. 3, Pre-op mean BMI=49.8±9.6 kg/m², 1 month post-op mean BMI=44.1±8.4 kg/m²). These findings indicate that the exosomes containing these specific proteins and miRNAs play a crucial role in the rapid and dramatic improvement of insulin sensitivity in the immediate period after bariatric surgery.

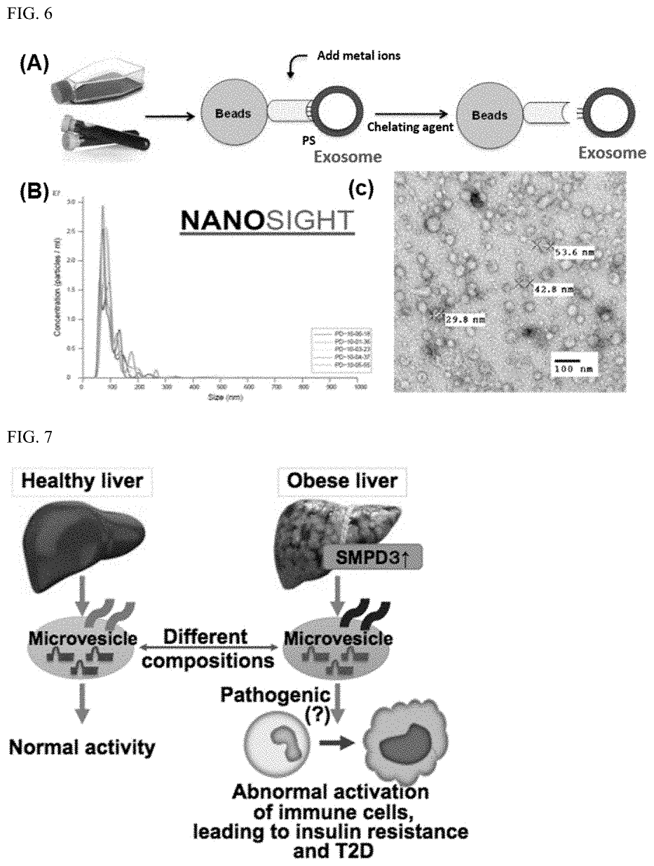

We have isolated exosomes by utilizing a novel affinity purification method using a phosphatidylserine (PS)-binding protein that uniquely and specifically interacts with a membrane of exosome (FIG. 6A, MagCapture™, WAKO). This technique has been verified for its capability to reliably isolate high purity exosomes. Isolated exosomes are analyzed by the Nanosight, a semi-automated, nanoparticle tracking analysis (NTA) method for their size and serum concentration measurement (FIG. 6B). For morphological analysis, we plan to perform Transmission Electron Microscopy (TEM) of the isolated exosomes (FIG. 6C). To our knowledge, this is the first demonstration of changes in serum exosome profile in obese subjects undergoing bariatric surgery.

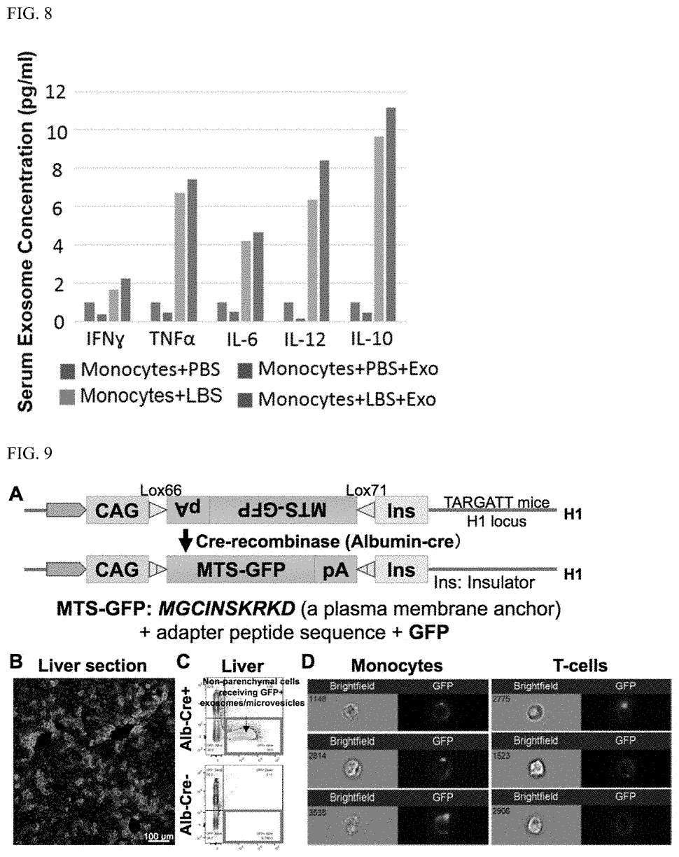

In another aspect, in obesity, hepatocyte-derived EVs become pathogenic and drive recipient immune cells, such as monocytes/macrophages, towards abnormal inflammation, leading to insulin resistance and T2D (See FIG. 7). We have established human monocyte isolation protocol using CD14 negative selection method (StemCell) and a reliable source for blood supply. We have optimized these experimental protocols and tested a number of experimental settings (including a number of different exosome doses) utilizing exosome samples from lean and metabolically healthy individuals (FIG. 8).To date, no in vivo model has been established to analyze specific tissue-derived EVs. To evaluate our hypothesis, we have recently developed a new mouse model in which EVs can selectively be labeled with a green fluorescent protein (GFP) in a tissue-specific manner under the Cre-LoxP system. By crossing the line with albumin-cre mice (Jackson Laboratory (JAX): 003574), we successfully labeled hepatocyte-derived EVs with GFP in vivo. By utilizing this mouse model, we investigated the role of hepatocyte-derived EVs becoming pathogenic and inducing inflammation during the course of obesity in vivo and in vitro. In addition, our study has indicated an involvement of hepatic Sphingomyelin Phosphodiesterase 3 (SMPD3, also known as nSMase2), an enzyme that generates ceramide and regulates RNA loading onto exosomes, in conferring the pro-inflammatory trait to the hepatocyte-derived exosomes under obese conditions. Thus, SMPD3 in hepatocytes plays a critical role in EV-mediated systemic inflammation and can be a therapeutic target for insulin resistance and T2D in obesity.

As mentioned above, there currently no tools/methods to isolate and analyze specific cell/tissue-derived EVs in vivo. We, therefore, initiated and established a novel mouse line (C57BL/6 strain) in which specific cell/tissue-derived EVs are selectively labeled with green fluorescent protein (GFP) tagged with a EV target sequence (ETS), which localizes the GFP to the microvesicular bilayer membrane. By utilizing the Lox66 and Lox71 sequences, the ETS tagged GFP gene (ETS-GFP) is “flipped” on when Cre-recombinase is expressed which, in turn, leads to expression of the ETS-GFP. Breeding this ETS-GFP line with Albumin-cre mice (ETS-GFp^{Alb-cre} mice), we successfully labeled EVs with GFP in a hepatocyte-specific manner. To our knowledge, this is a first mouse line that can analyze the tissue-specific EVs in vivo. In addition, the flow cytometry analysis (FCM) with non-parenchymal cells isolated from the liver revealed that the GFP-labeled EVs were taken up by several types of immune cells, including monocytes, macrophages, and T cells, which are albumin negative cells (FIGS. 9C and 9D). The GFP-positive signals indicated EVs from hepatocytes were transferred to non-parenchymal cells in the liver.

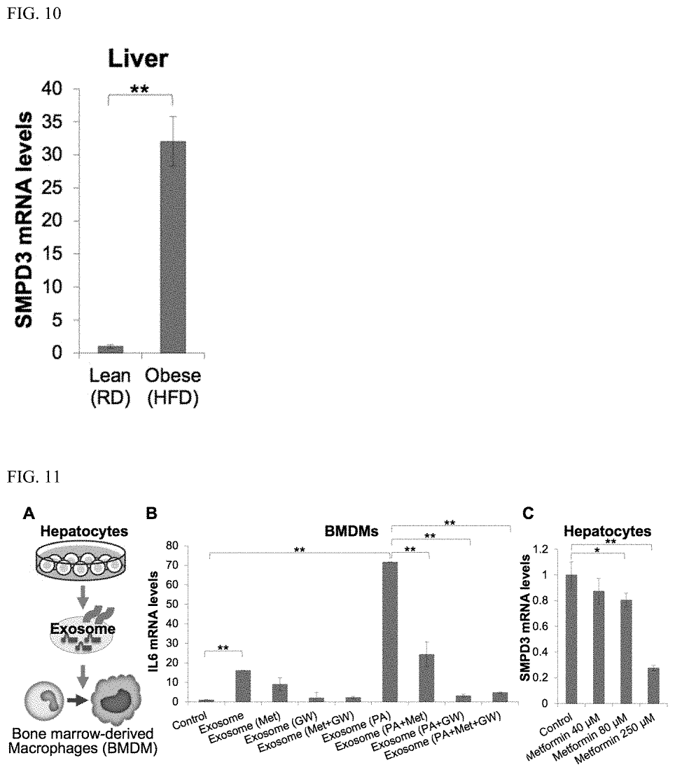

SMPD3 plays a critical role in EV secretion and affects components of EV such as RNA. We found that expression levels of SMPD3 are highly induced in obese liver (FIG. 10). Intriguingly, our studies showed that an exosome fraction (by a MagCapture Exosome Isolation kit from WAKO) isolated from cultured hepatocytes treated with palmitate, a pro-inflammatory fatty acid, caused higher inflammatory responses in primary murine macrophages (FIG. 11). Conversely, co-treatment of palmitate with a SMPD3 specific inhibitor (GW4869) strikingly attenuates the inflammatory effect of exosomes on macrophages. Importantly, co-treatment of palmitate with metformin, an anti-diabetic drug with anti-inflammatory effects while its molecular mechanism is still unclear, also diminished the inflammatory effect of exosomes on macrophages. GW4869 (CAS 6823-69-4) is a cell-permeable, potent, specific, non-competitive inhibitor of N-SMase (IC50=1 rat brain) that has been shown to inhibit 1) exosome secretion and 2) RNA loading onto exosomes. We also observed that metformin treatment suppresses SMPD3 expression in cultured hepatocytes (FIG. 11C). These results suggested three possibilities: first, SMPD3-dependent ceramide generation and/or RNA loading onto exosomes are crucial mechanisms in pro-inflammatory development of the hepatocyte-derived exosome fraction; second, SMPD3 is tightly linked with the pro-inflammatory feature of hepatocyte-derived exosomes in obesity; third, metformin exerts its anti-inflammatory effect by modulating functions of hepatocyte-derived exosomes.

Since inhibiting SMPD3 in hepatocytes diminished the inflammatory effect of exosomes, SMPD3-regulated components of exosomes are critical to the induction of inflammation. Two targets of SMPD3 actions are ceramide generation and RNA loading onto EVs, suggesting these regulated components play critical roles in the pro-inflammatory feature of hepatocyte-derived EVs.

Expression of SMPD3 is induced by palmitate exposure in cultured hepatocytes and in the obese liver. Since inhibition of SMPD3 enzymatic activity in hepatocytes reduces pro-inflammatory effects of exosomes on macrophages, reducing SMPD3 expression in the obese liver would reduce pro-inflammatory exosome secretion from the liver and alleviate local and systemic inflammation.

### EXAMPLES

### Example 1

To further establish that bariatric surgery directly affects the number and composition of serum exosomes in humans (FIG. 1), we plan to perform comprehensive analyses of serum exosome profile in obese adolescents in the immediate period after bariatric surgery (up to 3 months post-op) by expanding the sample number (n=40) to confirm that the surgery reduces serum exosome concentration.

Given that there are distinctive patterns of exosomal proteins and RNAs 1 month after surgery (FIGS. 4 and 5), we plan to further identify unique exosomal components that are affected by bariatric surgery. We will analyze protein contents of exosomes by performing proteomics with mass spectrometry. Once we identify proteins that are differentially expressed after surgery, we will validate the findings by performing Western Blot analysis. Additionally, we will increase sample numbers of the whole exosomal RNA-seq analysis to confirm our data above (FIG. 5) and further investigate specific RNAs that are differentially expressed post-op.

In parallel, the demographic and the clinical data will be collected, including patients' age, sex, race, height, weight, BMI, hemoglobin A1C (HgA1C), a fasting plasma glucose level (Gf), a fasting insulin level (If), lipid profile, hepatic profile and a C-reactive protein level (CRP) at each time point. Additionally, we plan to measure different cytokines, including IL-6, IL-10, TNF-alpha and adiponectin at each time point.

The mean serum exosome concentration at each time point (before, 1 and 3 months after surgery) and those will be compared using t-test or ANOVA, as appropriate. RNA-seq reads described above were processed and quantified using a web-based data analysis tool (http://www.genboree.org). Raw read counts were normalized, and a subsequent differential expression detection (p<0.05) was performed by the paired/matched design. We plan to validate these findings by performing quantitative polymerase chain reaction (qPCR). Then, those will be further investigated for their target site predictions by utilizing a miRNA database (MicroRNA.org). The demographic and clinical data will also be processed. Once collecting molecular data is completed, it will be correlated to the demographic and clinical data. This approach was implemented in order to achieve an unbiased approach in analyzing molecular data, in opposed to a targeted approach. To assess the improvement in insulin sensitivity after surgery, we plan to calculate established insulin sensitivity indices, including I/I_{f} and G_{f}/I_{f} at each time points.

These data will provide information of 1) the cellular origin of the exosomes that had the most significant impact after bariatric surgery, 2) potential molecular mechanisms of exosome function at target tissues to regulate insulin sensitivity, and 3) potential targets for novel biomarkers or treatments for insulin resistance. With TEM study, we anticipate to confirm that exosomes keep intact structures after bariatric surgery, despite the reduction in their concentration.

Identifying the cellular origin of exosomes has been challenging due to the heterogeneous nature of systemic exosomes. We anticipate to advance our understanding of the origin tissue of exosomes by analyzing exosomal proteins and RNAs, we may need further investigation. Therefore, we also plan to perform proteomics using exosomes isolated from various types of human cell, including hepatocytes (Corning), differentiated preadipocytes (Sigma) and myocytes (Sigma). Through this, we will be able to identify origin-tissue-specific exosome markers that can be used to detect cellular origins of distinctive serum exosome population.

### Example 2

Determine if the quantitative and/or compositional changes of exosomes affect hepatic insulin sensitivity. We chose to examine the hepatic insulin sensitivity as 1) liver is one of the major insulin target organs, and 2) it has been reported that hepatic insulin sensitivity improves within a week after bariatric surgery independent of weight loss. We will treat human primary hepatocytes (Corning) with 1) exosomes in a dose dependent manner, and 2) exosomes isolated from the study subjects before and 1, 3 months after bariatric surgery (for this, we will normalize exosome concentration). Then, we will perform in vitro assays to investigate the effect of exosome on hepatic insulin signaling which can be detected by insulin-induced phosphorylation levels insulin signaling molecules, including Irs1 and Akt, with Western blot analysis. Additionally, we will further investigate if exosomes modulate 1) insulin-induced suppression of glucose production, 2) expression level of messenger RNA (mRNA) of the genes regulating glucose metabolism such as Glucose-6-phosphatase (G6Pase), phosphoenolpyruvate carboxykinase 1 (PEPCK1), and Glucose transporter 2 (Glut2), and 3) expression level of mRNA that are potential targets for the exosomal RNA species identified above, such as Irs1, a target of miR-191, and Akt, a target of miR-150.

We anticipate that higher exosome concentration induces hepatic insulin resistance and/or exaggerated cytokine secretion in vitro. And the quantitative and/or the compositional changes of exosomes after bariatric surgery ameliorate those effects of exosomes and improve hepatic insulin sensitivity and/or immune derangement in vitro.

We hypothesize that the exosomes isolated from obese adolescents after bariatric surgery will behave more similarly to the exosomes from lean individuals (FIG. 1). Therefore, we plan to compare the data from this project to an age/sex/race similar lean group (recruitment is on-going). The next step is to define the role of the exosome system as a novel intercellular communication mode regulating insulin sensitivity by investigating origin and target tissues for each distinctive exosome subgroup. By analyzing exosomes contents, including proteins and RNAs, we will try to identify the origin tissues. We also plan to analyze the gene expression that are targeted by the identified exosomal RNAs described above, and conduct experiments utilizing antisense oligonucleotides (antimir) to induce or ameliorate the phenotypes caused by certain exosomal RNAs.

### Example 3

Determine if high-fat diet (HFD)-induced obesity affects quantity and components of hepatocyte-derived EVs in vivo. We will establish an experimental strategy to examine the quantity and components of tissue-specific EVs and determine the role of hepatocytes in their regulation. Determining the proportion of EVs secreted by each tissue/organ compared to body fluids/serum and whether this proportion changes in response to environmental factors such as exposure to HFD, is essential to understanding the bio-active properties of EVs. We are in a position to address this issue with our newly established mouse model. The GFP fluorescence, moreover, permits isolation of tissue-specific EVs by nanoFACS, for Omics analyses of their components. We expect a subpopulation of hepatocyte-derived EVs with defined changes in RNA and lipid components is increased in serum, in response to HFD feeding.

The ETS-GF^{Alb-cre} mice and controls (ETS-GFP) will be placed on regular diet (RD: 12.7% fat, 58.5% carbohydrate, 28.8% protein; LabDiet) (males (n=8) and females (n=8)) or HFD (60% fat, 20% carbohydrate, 20% protein; Research Diets # D12492) (males (n=8) and females (n=8)) at 4 weeks of age for 2, 4, and 8 weeks. Body weight, blood glucose, plasma insulin, and liver enzyme activity will be measured to confirm the effect of HFD feeding in this cohort. EVs will be isolated from serum through the size-exclusion chromatography columns (iZON) that can purify EVs, including exosome fractions. Concentrations of total and GFP-labeled EVs in serum will be analyzed by the Nanosight equipped with a blue laser detecting GFP signal, which is currently a gold standard system to analyze microvesicular concentration and size. By analyzing the concentration of GFP-labeled hepatocyte-derived EVs in serum, we will calculate how many ETS-GFP^{Alb-cre} mice are required for collecting the GFP-labeled hepatocyte-derived EVs for the Omics analyses. Our preliminary study indicates that 1×10¹¹ EVs are sufficient for total RNA seq and lipidomics, respectively.

### Example 4

Determine if obesity changes cell- and sub-types of immune cells receiving hepatocyte-derived EVs in vivo. In obesity, there are changes in compositions of cell- and sub-types of immune cells towards more pro-inflammatory cells. For instance, during HFD feeding, there is an increased number of recruited hepatic macrophages in the liver and activation of Kupffer cells to a more inflammatory or M1 state. As a first step, we will investigate cell- and sub-types of immune cells in the liver that are receiving GFP-labeled hepatocyte-derived EVs and evaluate whether there are differences in cell- and sub-types between lean and obese conditions. We will also compare the sub-types between GFP positive and negative macrophages in obesity to assess if the GFP positive macrophages become more inflammatory/M1 state. We believe there will be higher numbers of GFP-positive immune cells in the obese liver and these GFP-positive immune cells are more pro-inflammatory than the corresponding GFP-negative cells.

We will first perform FCM, as shown in FIG. 9C, with livers from mice prepared as described above. For FCM, single cell suspension will be prepared with collagenase treatment, followed by Percoll gradient purification that removes hepatocytes and red cell lysis (FIG. 9C and 9D). The non-parenchymal cells will be subject to the FCM with antibodies markers for monocytes, macrophages, neutrophils, dendritic cells, T-cells, and B-cells. In addition, we will perform the imaging flow cytometer analysis (ImageStreamX) to confirm that the immune cells graft GFP-labeled hepatocyte-derived EVs (FIG. 9D). To further confirm the cell- and sub-types of immune cells receiving hepatocyte-derived EVs, we will perform the single-cell RNA seq analysis with GFP-positive and negative immune cells sorted from the non-parenchymal cells' population from the livers of mice prepared above (ETS-GFP^{Alb-cre}, RD and HFD for 8 weeks).

### Example 5

Determine pro-inflammatory effects of obesity-driven, hepatocyte-derived EVs. As shown in FIG. 9D, we observed that immune cells in the liver uptake GFP-labeled hepatocyte-derived EVs in vivo. In this example, we will assess if hepatocyte-derived EVs from obese mice remain inflammatory when transferred to lean mice. We believe that hepatocyte-derived EVs isolated from obese mice are more pro-inflammatory than those from lean mice.

We will first conduct pilot EV transfer experiments to evaluate the efficiency of EV uptake in recipient immune cells in the liver. In these experiments, we will prepare GFP-positive EVs from ETS-GFp^{Alb-cre} mice fed RD and administer to recipient wild-type CD1 mice, which have white fur, through tail vein injection and detect GFP fluorescence by the IVIS Imaging System at 6, 12, 24, and 48 hours post GFP-positive EV transfer. We will also administer the GFP-positive EVs to wild-type C57BL/6 mice and analyze GFP signal in immune cells in the liver at 6, 12, 24, and 48 hours post EV transfer (see FIG. 9C and 9D). Based on these analyses, we will estimate a half-life of GFP signal in the recipient mice post EV transfer and can administer GFP-positive EVs isolated from ETS-GFP^{Alb-cre} mice fed RD and HFD into C57BL/6 mice multiple times (at least three times). GFP positive immune cells will be subsequently isolated for FCM and single-cell RNA seq analyses, as described above, to compare the pro-inflammatory effect on immune cells between hepatocyte-derived EVs isolated from lean and obese mice.

We predict that the population and number of GFP-positive immune cells in the liver are quite different between lean and obese conditions in ETS-GFP^{Alb-cre} mice. We also predict that the population of GFP-positive immune cells can be differentiated from that of GFP-negative immune cells in obese livers of ETS-GFP^{Alb-cre} mice. This trend would be confirmed by the EV transfer experiment described above. By analyzing results of single-cell RNA seq with bioinformatics, gene expression profiling may be more predictive of the various subpopulation. In the transfer experiments described above, since it is possible that specific fractions of EVs, e.g., exosomes, may play a critical role in the induction of inflammation, EVs will be size-fractionated (size-exclusion chromatography columns) and assessed.

### Example 6

Determine the role of SMPD3 in hepatocyte-induced local and systemic inflammation. As shown above, SMPD3 plays a critical role in EV secretion and affects components of EVs such as RNA. We found that expression levels of SMPD3 are highly induced in obese liver (FIG. 10). Intriguingly, our studies showed that an exosome fraction (by a MagCapture Exosome Isolation kit from WAKO) isolated from cultured hepatocytes treated with palmitate, a pro-inflammatory fatty acid, caused higher inflammatory responses in primary murine macrophages (FIG. 11). Conversely, co-treatment of palmitate with a SMPD3 specific inhibitor (GW4869) strikingly attenuates the inflammatory effect of exosomes on macrophages. Importantly, co-treatment of palmitate with metformin, an anti-diabetic drug with anti-inflammatory effects while its molecular mechanism is still unclear, also diminished the inflammatory effect of exosomes on macrophages. We also observed that metformin treatment suppresses SMPD3 expression in cultured hepatocytes (FIG. 11C). These results suggested three possibilities: first, SMPD3-dependent ceramide generation and/or RNA loading onto exosomes are crucial mechanisms in pro-inflammatory development of the hepatocyte-derived exosome fraction; second, SMPD3 is tightly linked with the pro-inflammatory feature of hepatocyte-derived exosomes in obesity; third, metformin exerts its anti-inflammatory effect by modulating functions of hepatocyte-derived exosomes. In this example, we will determine the role of hepatic SMPD3 in EV-mediated local and systemic inflammatory responses in obesity.

Determine the hepatic SMPD3-dependent exosome components that induce inflammatory responses. Since inhibiting SMPD3 in hepatocytes diminished the inflammatory effect of exosomes, SMPD3-regulated components of exosomes are critical to the induction of inflammation. Two targets of SMPD3 actions are ceramide generation and RNA loading onto EVs, suggesting these regulated components play critical roles in the pro-inflammatory feature of hepatocyte-derived EVs. In this example, we will mainly analyze the exosome fraction as we observed its profound pro-inflammatory features (FIG. 11). We believe that ceramide and/or RNA in exosomes and their receptors, toll-like receptors (TLR4) and TLR7, induce inflammation in macrophages. We will culture bone marrow-derived macrophages (BMDM) from TLR4-deficient (JAX: 007227), TLR7-deficient (JAX: 008380), and control mice. We will also prepare exosomes collected from primary mouse hepatocyte treated with palmitate, metformin, GW4869, singly and in combinations, and determine if TLR4 and/or TLR7 inflammatory pathways in macrophages are induced in response to the isolated hepatocyte-derived exosomes. Lipidomics and RNA seq analyses followed by bioinformatics analysis will be performed, to determine the effects of the treatments of palmitate, metformin, and GW4869 on components of exosomes. As indicated above, 1×10¹¹ exosomes are sufficient for total RNA seq and lipidomics, respectively.

Determine if targeting hepatic SMPD3 can alleviate local and systemic inflammation. Expression of SMPD3 is induced by palmitate exposure in cultured hepatocytes and in the obese liver. Since inhibition of SMPD3 enzymatic activity in hepatocytes reduces pro-inflammatory effects of exosomes on macrophages, it is possible that reducing SMPD3 expression in the obese liver would reduce pro-inflammatory exosome secretion from the liver and alleviate local and systemic inflammation. We believe that suppression of SMPD3 expression in the obese liver would be beneficial to reduce local and systemic inflammation and improve glucose metabolism in obesity.

To further investigate the impact of suppressing liver-specific SMPD3 expression, we will utilize an Adeno-associated virus (AAV) 8-mediated gene transfer system to specifically express siRNAs against SMPD3 (siSMPD3) in the liver under the regulation of the albumin promoter (VECTOR BIOLABS). Since the AAVs exhibit minimal pathogenicity and have significantly longer transgene expression, we are able to assess the effect of sustained suppression of SMPD3 without severe induction of generalized inflammatory responses. Our data confirmed the specific expression of our gene of interest in the liver over 27 weeks post-AAV infection without inflammatory responses (data not shown). We will introduce AAV8 (1.0×10¹² genomic copy/mouse) carrying the siSMPD3 (males (n=8) and females (n=8)) or control siRNAs (males (n=8) and females (n=8)) in ETS-GFp^{Alb-cre} mice at 4 weeks of age and place these mice on HFD for 8 weeks. Inflammatory status of these mice will be assessed by both standards analyses, including inflammatory cytokines levels in plasma and activation levels of inflammatory kinases such as c-Jun N-terminal kinase (JNK) in the liver, and our newly established methods with the ETS-GFP^{Alb-cre} mice described above. We will also measure plasma insulin, blood glucose, and hemoglobin A1c (HbA1c) levels in evaluating the effect of hepatic SMPD3 knockdown on glucose metabolism under obese conditions. Two cohorts will be analyzed, based on biostatistical advice from our internal advisors.

We predict that the SMPD3-regulated ceramides and/or RNAs in hepatocyte-derived EVs activate TLR pathways and induces inflammatory responses in macrophages. If TLR4- and TLR7-pathways are dispensable, we will analyze other TLR pathways by utilizing TLR2-, TLR3-, or Myeloid differentiation primary response 88 (Myd88)-deficient macrophages. If other TLR pathways are also dispensable, we will additionally assess other pathogen sensors such as components of the NLRP3 inflammasome and RNA silencing. It is possible that protein components of EVs trigger activation of macrophages. Hence, if it is difficult to identify the ceramide- and/or RNA-relevant pathways activating recipient macrophages, we will perform proteomic analyses to identify specific components of exosomes isolated from hepatocytes treated with GW4869 or its control. We also predict that suppression of hepatic SMPD3 would reduce inflammation and improve glucose metabolism in obesity. In this study, we will utilize the AAV-mediated SMPD3 knockdown model. However, if technical difficulties surface, we will proceed to generate hepatocyte-specific SMPD3 conditional knockout mice with embryonic stem (ES) cell lines. This different approach will still permit us to evaluate the role of hepatic SMPD3 in the pathogenesis of insulin resistance and T2D in obesity.

Each of the following references is incorporated herein by reference in its entirety.

Hales, C. M., Fryar, C. D., Carroll, M. D., Freedman, D. S. & Ogden, C. L. Trends in Obesity and Severe Obesity Prevalence in US Youth and Adults by Sex and Age, 2007-2008 to 2015-2016. JAMA, (2018).

Batterham, R. L. & Cummings, D. E. Mechanisms of Diabetes Improvement Following Bariatric/Metabolic Surgery. Diabetes Care 39, 893-901, (2016).

Valadi, H. et al. Exosome-mediated transfer of mRNAs and microRNAs is a novel mechanism of genetic exchange between cells. Nat Cell Biol 9, 654-659, (2007).

Huang-Doran, I., Zhang, C. Y. & Vidal-Puig, A. Extracellular Vesicles: Novel Mediators of Cell Communication In Metabolic Disease. Trends Endocrinol Metab 28, 3-18, (2017).

Xu, R., Greening, D. W., Zhu, H. J., Takahashi, N. & Simpson, R. J. Extracellular vesicle isolation and characterization: toward clinical application. J Clin Invest 126, 1152-1162, (2016).

Ying, W. et al. Adipose Tissue Macrophage-Derived Exosomal miRNAs Can Modulate In Vivo and In Vitro Insulin Sensitivity. Cell 171, 372-384 e312, (2017).

Thomou, T. et al. Adipose-derived circulating miRNAs regulate gene expression in other tissues. Nature 542, 450-455, (2017).

Wang, L. et al. Exosomes derived from pancreatic cancer cells induce insulin resistance in C2C12 myotube cells through the PI3K/Akt/FoxO1 pathway. Sci Rep 7, 5384, (2017).

Deng, Z. B. et al. Adipose tissue exosome-like vesicles mediate activation of macrophage-induced insulin resistance. Diabetes 58, 2498-2505, (2009).

Hotamisligil, G. S. Foundations of Immunometabolism and Implications for Metabolic Health and Disease. Immunity 47, 406-420, (2017).

Osborn, 0. & Olefsky, J. M. The cellular and signaling networks linking the immune system and metabolism in disease. Nat Med 18, 363-374, (2012).

Samuel, V. T. & Shulman, G. I. Mechanisms for insulin resistance: common threads and missing links. Cell 148, 852-871, (2012).

Inge, T. H. et al. Long-term outcomes of bariatric surgery in adolescents with severe obesity (FABS-5+): a prospective follow-up analysis. Lancet Diabetes Endocrinol 5, 165-173, (2017).

Lykken, E. A. & Li, Q. J. The MicroRNA miR-191 Supports T Cell Survival Following Common gamma Chain Signaling. J Biol Chem 291, 23532-23544, (2016).

Wu, S. J., Chen, J., Wu, B., Wang, Y. J. & Guo, K. Y. MicroRNA-150 enhances radiosensitivity by inhibiting the AKT pathway in NK/T cell lymphoma. J Exp Clin Cancer Res 37, 18, (2018).

Vu, P. Y. et al. A homozygous balanced reciprocal translocation suggests LINC00237 as a candidate gene for MOMO (macrosomia, obesity, macrocephaly, and ocular abnormalities) syndrome. Am J Med Genet A 158A, 2849-2856, (2012).

Nakai, W. et al. A novel affinity-based method for the isolation of highly purified extracellular vesicles. Sci Rep 6, 33935, (2016).

Nakamura, T. et al. A critical role for PKR complexes with TRBP in Immunometabolic regulation and eIF2alpha phosphorylation in obesity. Cell Rep 11, 295-307, (2015).

Robinson, M. D., McCarthy, D. J. & Smyth, G. K. edgeR: a Bioconductor package for differential expression analysis of digital gene expression data. Bioinformatics 26, 139-140, (2010).

George, L. et al. Surrogate estimates of insulin sensitivity in obese youth along the spectrum of glucose tolerance from normal to prediabetes to diabetes. J Clin Endocrinol Metab 96, 2136-2145, (2011).

Gazala, S. A. et al. Sleeve Gastrectomy Improves Glycemia Independent of Weight Loss by Restoring Hepatic Insulin Sensitivity. Diabetes, (2018).

Wild S, Roglic G, Green A, Sicree R and King H. Global prevalence of diabetes: estimates for the year 2000 and projections for 2030. Diabetes Care. 2004;27:1047-53.

Hotamisligil G S. Inflammation, metaflammation and immunometabolic disorders. Nature. 2017; 542:177-185.

Schorey J S, Cheng Y, Singh P P and Smith V L. Exosomes and other extracellular vesicles in host-pathogen interactions. EMBO Rep. 2015; 16:24-43.

Huang-Doran I, Zhang C Y and Vidal-Puig A. Extracellular Vesicles: Novel Mediators of Cell Communication In Metabolic Disease. Trends in endocrinology and metabolism: TEM. 2017; 28:3-18.

Kosaka N, Yoshioka Y, Fujita Y and Ochiya T. Versatile roles of extracellular vesicles in cancer. J Clin Invest. 2016; 126:1163-72.

Zacharias D A, Violin J D, Newton A C and Tsien R Y. Partitioning of lipid-modified monomeric GFPs into membrane microdomains of live cells. Science. 2002; 296:913-6.

Shen B, Wu N, Yang J M and Gould S J. Protein targeting to exosomes/microvesicles by plasma membrane anchors. J Biol Chem. 2011; 286:14383-95.

Zhang Z and Lutz B. Cre recombinase-mediated inversion using lox66 and lox71: method to introduce conditional point mutations into the CREB-binding protein. Nucleic Acids Res. 2002; 30:e90.

Morales-Kastresana A, Telford B, Musich T A, McKinnon K, Clayborne C, Braig Z, Rosner A, Demberg T, Watson D C, Karpova T S, Freeman G J, DeKruyff R H, Pavlakis G N, Terabe M, Robert-Guroff M, Berzofsky J A and Jones J C. Labeling Extracellular Vesicles for Nanoscale Flow Cytometry. Sci Rep. 2017; 7:1878.

Zincarelli C, Soltys S, Rengo G and Rabinowitz J E. Analysis of AAV serotypes 1-9 mediated gene expression and tropism in mice after systemic injection. Mol Ther. 2008; 16:1073-80.

What is claimed is:

1. A method for treating metabolic disorders in a subject in need thereof, the method comprising
isolating circulating extracellular vesicles (EV) from a healthy donor or from media of cultured cells, and
administering the EVs to the subject, under conditions sufficient to treat metabolic disorders in the subject.

2. The method of claim 1, wherein the metabolic disorders is an obesity-associated metabolic disease selected from insulin resistance, type-2 diabetes, fatty liver diseases, cardiovascular disease, atherosclerosis, and/or Alzheimer's disease.

3. The method of claim 1, wherein the EVs comprise miR-191, miR-150, LINC00237, and/or SMPD3.

4. The method of claim 1, wherein the EVs act by a direct interaction with insulin target tissues and/or by modulation of immune function.

5. The method of claim 4, wherein the insulin target tissues are selected from the group consisting of liver, adipose tissue, muscle, and combinations thereof.

6. The method of claim 1, wherein the EVs are isolated by an affinity-based isolation procedure using a phosphatidylserine (PS)-binding protein or by size-exclusion chromatography.

7. The method of claim 2, wherein insulin resistance is measured by measuring blood glucose, hemoglobin A1c, and/or insulin.

8. The method of claim 7, further comprising measuring blood glucose, hemoglobin A1c, and/or insulin before and after the administration of the EVs.

9. The method of claim 4, further comprising assaying the effect on immune function by measuring circulating levels of cytokines selected from the group consisting of IL-6, TNF-α, and IL-10 before and after the administration of the exosomes.

10. The method of claim 1, wherein the exosomes are administered at a dose and/or concentration similar to levels in healthy subjects.

11. The method of claim 1, wherein the healthy donor exhibits at least one of a body mass index (BMI) of less than about 25, normal blood glucose level, and/or no symptoms of fatty liver disease.

12. The method of claim 1, wherein the cultured cells are hepatocytes.

13. The method of claim 1, wherein the EV is an exosome.

14. A method for early detection of metabolic risks associated with obesity, the method comprising
obtaining circulating extracellular vesicles (EVs) from a subject,
measuring a concentration of EVs and/or a level of miR-191, miR-150, LINC00237, and/or sphingomyelin phosphodiesterase 3 (SMPD3) in the EVs,
comparing the concentration of EVs and/or the level of miR-191, miR-150, LINC00237, and/or SMPD3 in the EVs with the EV concentration and/or level of miR-191, miR-150, LINC00237, and/or SMPD3 in EVs from a healthy control, whereby
i) if the concentration of the EVs is greater than the healthy control;
ii) if the level of miR-191 is greater than the healthy control;
iii) if the level of miR-150 is less than the healthy control;
iv) if the level of LINC00237 is less than the healthy control;
v) if the level of SMPD3 is greater than the healthy control, the subject exhibits metabolic risks associated with obesity.

15. The method of claim 14, further comprising treating the subject found to exhibit metabolic risks associated with obesity.

16. The method of claim 15, wherein the treatment comprises neutralizing or depleting an amount of the EVs, or a subset thereof, such that development of metabolic diseases is ameliorated or prevented.

17. The method of claim 14, wherein the metabolic risks comprise an obesity-associated metabolic disease selected from insulin resistance, type-2 diabetes, fatty liver diseases, cardiovascular disease, atherosclerosis, and/or Alzheimer's disease.

18. A method for treating insulin resistance and/or type-2 diabetes, the method comprising administering an inhibitor of sphingomyelin phosphodiesterase 3 (SMPD3) to a subject in need thereof, under conditions sufficient to treat insulin resistance and/or type-2 diabetes.

19. The method of claim 18, wherein the inhibitor of sphingomyelin phosphodiesterase 3 (SMPD3) is GW4869, metformin, or siRNA directed to SMPD3 mRNA.

20. The method of claim 19, wherein metformin decreases the expression of SMPD3.

21. The method of claim 18, wherein SMPD3 stimulates ceramide generation and RNA loading into extracellular vesicles (EVs).

22. The method of claim 18, wherein inhibition of SMPD3 reduces pro-inflammatory exosome secretion, decreases local and systemic inflammation, and improves glucose metabolism.
